(19) United States
(12) Patent Application Publication
(10) Pub. No.: US 2022/0395198 A1
(43) Pub. Date: Dec. 15, 2022

(54) SELF-POWERED STRETCHABLE SWEAT SENSORS

(71) Applicant: City University of Hong Kong, Hong Kong (HK)

(72) Inventors: Xinge YU, Hong Kong (HK); Xingcan HUANG, Hong Kong (HK); Yiming LIU, Hong Kong (HK)

(21) Appl. No.: 17/837,042

(22) Filed: Jun. 10, 2022

Related U.S. Application Data
(60) Provisional application No. 63/210,076, filed on Jun. 14, 2021.

(51) Int. Cl. A61B 5/145; H01M 6/32; A61B 5/00

(52) U.S. Cl. CPC A61B 5/14517; A61B 5/14546; H01M 6/32; A61B 5/6833; A61B 5/0022; A61B 2560/0214

(57) ABSTRACT

The present invention provides a flexible and stretchable wearable electronic device system. The system includes a sweat-activated battery that has an anode and a cathode and a dry electrolyte-impregnated carrier in contact with the anode and the cathode. A sweat-absorbing layer is in contact with the dry electrolyte-impregnated carrier. An adhesive layer is provided for attaching the sweat-activated battery to the skin of a user. A flexible electronic device such as a sweat sensor or a lighting element is connected to the sweat-activated battery and is powered by the sweat-activated battery.

### TECHNICAL FIELD

The invention is relating to a wearable, flexible sweat-activated battery capable of powering flexible electronics, including lighting LEDs and wirelessly, continuously monitoring sweat components.

### BACKGROUND

Sweat sensors are used in a variety of situations where a user's output of sweat needs to be closely monitored. These situations include athletic pursuits, medical monitoring, as well as construction and outdoor work. In addition to monitoring the sweat output, such sensors may also monitor the chemical composition of sweat in order to provide information on the health status of the wearer including glucose levels and salt/sodium levels.

In order for sweat sensors to be widely deployed for health monitoring purposes, the sensors need to be convenient to wear for the users. They must also be low-cost and have a ready supply of energy to power the sensors. Thus, there is a need in the art for sweat sensors that move and flex with the user's motions and which can provide energy to power the sensors.

### SUMMARY OF THE INVENTION

The sweat-activated stretchable battery (SASB) is an entirely flexible device fabricated on a soft and deformable substrate. The battery may include two thin metal sheets, for example, copper (Cu) and zinc (Zn), acting as cathode and anode, respectively. There are two fabric blocks (for example, nylon), each containing a biocompatible chemical crystal, for example, including copper (II) sulfate (CuSO₄) and potassium chloride (KCl). Fabric containing KCl and CuSO₄ particles are wrapped with Zn and Cu metal sheets, respectively. These fabrics act as the corresponding electrolyte in a galvanic cell once they absorb a fluid such as sweat. A relatively thick absorbing layer (for example, absorbent cotton) with high absorbing capability is impregnated with KCl powder and is responsible for absorbing sweat efficiently. This layer acts as the salt bridge and provides a flow of ions once it absorbs sweat.

The battery working principle is based on the chemical reaction between Zn and CuSO₄. Simultaneously, two fabric pieces act as the electrolyte containing ions, and the absorbing layer (with KCl) serves as the salt bridge allowing ion transfer between two fabrics. The electrolytic reaction starts immediately after the absorbing layer absorbs the sweat, allowing ions to flow in the system.

The reliable performance of the battery has been safely demonstrated by powering flexible lighting electronics attached to the arm of a runner. In this case, the battery is activated by the secreted sweat from the human body to power a lighting LED series. The flexible battery can power electronic lighting for more than 8 hours. In addition, the flexibility and portability of the device make it easy to use during running or other physical activities. Due to these characteristics, the device perfectly addresses safety concerns for a running individual in the dark by alerting others to his/her presence.

There are abundant chemicals in the human sweat that reflect an individual's health status. Taking this into consideration, a smart stretchable microelectronic device is developed and powered by the four sweat-activated battery (FSASB) cells to wirelessly analyse and monitor the concentration of three different sweat biomarkers: pH, Na+, and glucose. The microelectronic device contains a microfluidic system for absorbing human perspiration with a controlled flow rate. The performance of the flexible microelectronic device containing sweat sensors is examined over different concentrations and over time. Results show stable and accurate measurements.

In one aspect, the present invention provides a flexible and stretchable electronic device system including a sweat-activated battery having an anode and a cathode. A dry electrolyte-impregnated carrier is in contact with the anode and the cathode. A sweat-absorbing layer is in contact with the dry electrolyte-impregnated carrier. An adhesive layer attaches the sweat-activated battery to the skin of a user. A flexible electronic device is connected to the sweat-activated battery, and is powered by the sweat-activated battery.

In another aspect, the dry electrolyte-impregnated carrier includes one or more of CuSO₄ or KCl.

In another aspect, the sweat-absorbing layer is impregnated with a dry electrolyte.

In another aspect, the sweat-absorbing layer is impregnated with KCl such that the sweat-absorbing layer acts as a salt bridge in a galvanic cell.

In another aspect, the flexible electronic device is an LED lighting device.

In another aspect, the flexible electronic device is a sweat sensor.

In another aspect, the sweat sensor includes detectors for monitoring pH, sodium levels, and glucose levels.

In another aspect, the system includes a deformable carrier housing the sweat-activated battery.

In another aspect the deformable carrier is polydimethylsiloxane.

In another aspect, the system further includes a wireless transmitter for transmitting signals to a wireless receiver.

### BRIEF DESCRIPTION OF THE DRAWINGS

FIGS. 1(a)-1(e) depict a sweat-activated battery and its components according to an embodiment;

FIGS. 2(a)-2(d) are photographs of an example of a fabricated sweat-activated battery and its components according to an embodiment;

FIG. 3 depicts operation of the sweat-activated battery of FIG. 1;

FIG. 4 schematically depicts fabrication of the sweat-activated battery of FIG. 1;

FIG. 5 shows optical images of battery cell encapsulation;

FIGS. 6(a)-6(h) depict electrical characteristics of the battery of FIG. 1;

FIG. 7 depicts the voltage output of the battery of FIG. 1;

FIGS. 8(a)-8(b) depict test results for the battery of FIG. 1;

FIGS. 9(a)-9(c) depict stretching and tests of stretched batteries of FIG. 1;

FIGS. 10(a)-10(c) depict deformation of the battery of FIG. 1 and the electrical response during deformation;

FIGS. 11(a)-11(g) depict the application of the battery of FIG. 1 to an LED array for a safety device;

FIGS. 12(a)-12(j) depict the application of the battery of FIG. 1 to a sweat sensor device;

FIGS. 13(a)-13(c) depict open circuit voltage responses for glucose, sodium, and pH sensors;

FIGS. 14(a)-14(c) depicts the anti-interference capabilities of glucose, sodium, and pH sensors;

FIGS. 15(a)-15(b) depict a sweat sensor powered by plural batteries of FIG. 1;

FIGS. 16(a)-16(c) show photos of a prototype sweat sensor;

FIGS. 17(a)-17(d) depict testing of a sweat sensor and the results;

FIG. 18 shows the transmission distance of the sweat sensor powered by plural batteries of FIG. 1;

FIG. 19 shows photographs of the sweat sensor powered by the battery of FIG. 1 mounted on various body parts.

### DETAILED DESCRIPTION

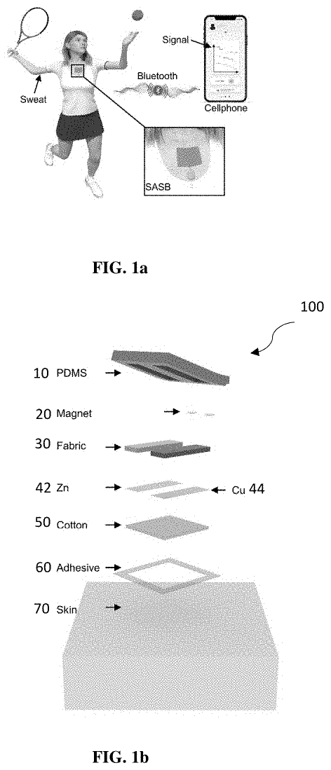

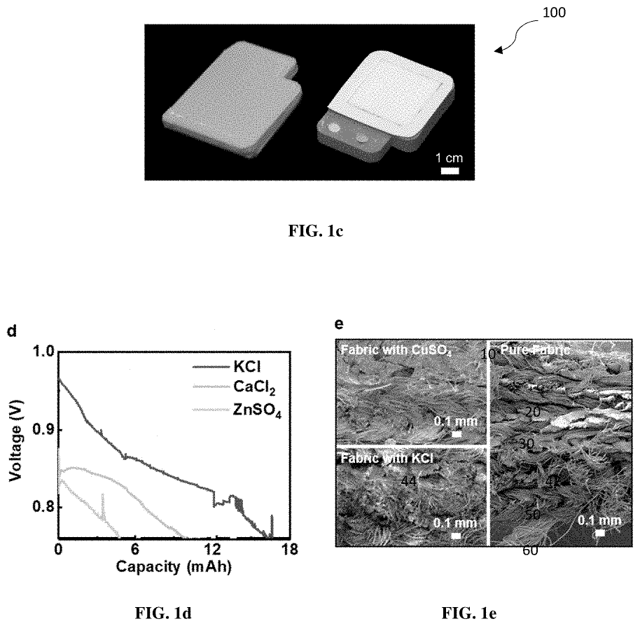

Turning to the drawings in detail, FIG. 1(b) depicts a stretchable battery 100 according to an embodiment. The stretchable battery includes plural layers. A flexible, non-toxic, and biocompatible substrate 10 is used as a carrier/base. The substrate 10 may be a flexible polymer such as polydimethylsiloxane (PDMS; crosslink: PDMS, 1:30) Two small circular magnets 20 are embedded in the substrate 10. The magnetic sheets 10 may be made with a diameter of approximately 5 mm and a thickness of 1 mm and are physically wired to the battery's electrodes. The purpose of using these two magnets is to ease powering of external electronics.

Two electrolyte carriers 40 are impregnated with electrolytes to be used in the electrolytic reaction in the battery. For example, the carriers 40 may be fabric that has been immersed in copper sulfate (CuSO₄) and potassium chloride (KCl) solutions, respectively, then dried at an elevated temperature, for example, 120° C. These electrolytes are exemplary, other electrolytes may also be used. As a result, the two electrolytes are crystallized and deposit into the fabrics. This process may be repeated for several cycles to increase the mass of stored electrolyte particles in the carrier fabric until a desired mass is obtained. Free movement of ions (Zn²⁺, Cu²⁺, and Cl⁻) in the carrier becomes possible upon absorbing sweat and dissociating in the sweat solution. Scanning electron microscope (SEM) images of the carriers before and after immersion in the two afore-mentioned solutions are presented in FIG. 1(e), which depicts the carrier's ability to store crystallized chemicals. The selected fabrics, for example, nylon-based fabrics, are highly biocompatible, flexible, and well-suited for storing crystallized chemicals. In an example, CuSO₄— and KCl— impregnated fabric blocks each weighing approximately 0.60 g are used, which includes approximately 0.37 g of electrolyte particles and approximately 0.23 g of fabric carrier.

Two thin electrode sheets 42 and 44 are placed into contact with the electrolyte-impregnated carrier 30. The electrodes may be selected from a variety of electrically-conductive materials that participate in the overall electrolytic reaction of the battery. For example, copper (Cu) and zinc (Zn) metal sheets, with a thickness of approximately 0.08 mm, may be integrated with the fabric impregnated with CuSO₄ and KCl, and form the cathode and anode, respectively. When the fabric impregnated with KCl particles absorbs sweat, Zn is oxidized to Zn²⁺ and enters the electrolyte fabric, while the Cl⁻ ions dissociate from KCl in the aqueous environment, compensating for the charge difference from the zinc ions.

Zn (s)→Zn²⁺ (aq)+2e⁻ (Oxidation half reaction)

In the other impregnated fabric, Cu²⁺ ions plate onto the copper metal sheet taking up electrons from the external circuit.

Cu²⁺ (aq)+2e⁻→Cu (s) (Reduction half reaction)

As a result, the overall reaction occurs between Zn and CuSO₄:

Zn (s)+Cu²⁺ (aq)→Zn²⁺ (aq)+Cu (s)

A relatively thick layer of absorbent material 50 impregnated with KCl powder is used as the water absorption layer in order to absorb perspiration. For example, a cotton layer (thickness, approximately 1.6 mm; mass, approximately 0.18 g cotton/0.22 g KCl) impregnated with KCl or other electrolyte material may be used. The cotton layer may also be impregnated with KCl by immersion in a KCl solution and drying at 120° C. for several cycles. As a result, absorbent layer 50 contains KCl particles through a process similar to the one previously applied to layer 30. Absorbent layer 50 acts as a salt bridge, which provides the flow of ions in the battery reaction.

In general, the battery is positioned on the skin of the wearer. As the wearer sweats, the absorbent layer 50 absorbs sweat; the sweat reaches the inner part of the battery, including fabric layers 30. The sweat dissolves the crystallized electrolyte particles, allowing them to become aqueous ions. These aqueous ions flow through the system, resulting in a chemical reaction between Zn and CuSO₄ as schematically depicted in FIG. 3.

An adhesive layer 60 is added to the device to facilitate wearing and provide sustainable contact for perspiration absorption from the human body.

The best performance of the battery is observed when the KCl has been used as the saturated electrolyte for the impregnated fabric integrated with the Zn metal sheet. For this example, the battery can provide an energy capacity as high as 16.6 mAh while the output voltage drops from 0.97V to 0.76V. (FIG. 1(d))

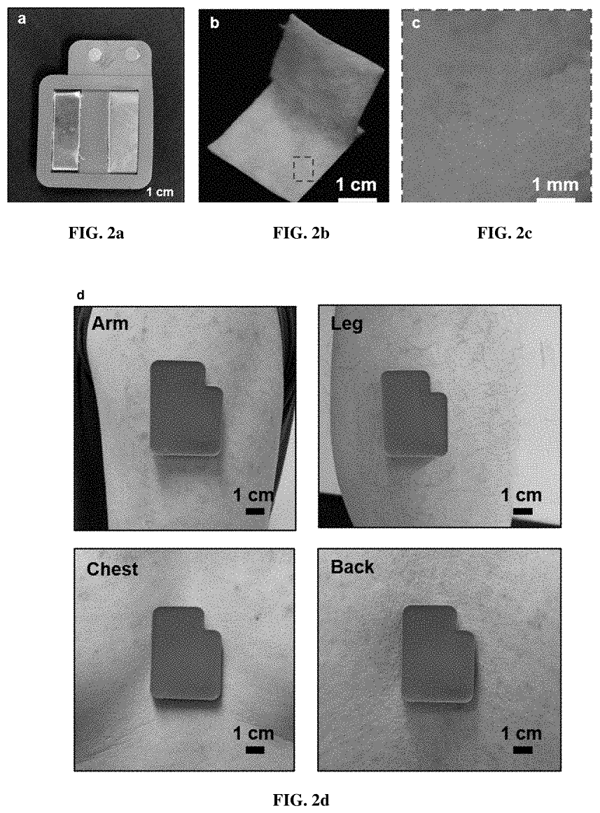

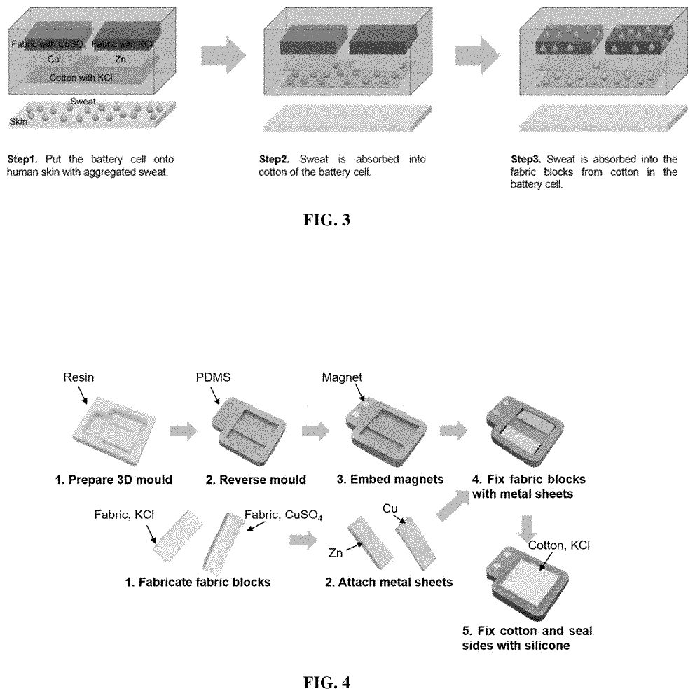

The final device 100, as seen in FIG. 1(c) and FIG. 2(d) is a small, soft, thin, and flexible patch. Additionally, it is a wearable, easy-to-use, and biocompatible device. The overall size of the device in this example is approximately 6.3 cm×5 cm with a thickness of approximately 6 mm. An illustration of a person wearing a flexible electronic device powered by the sweat-activated battery for wireless sweat components monitoring is illustrated in FIG. 1(a). Optical images of the inner side of the battery, water absorption layer, and battery cell mounted onto different parts of the human body are shown in FIG. 2(a)-(d).

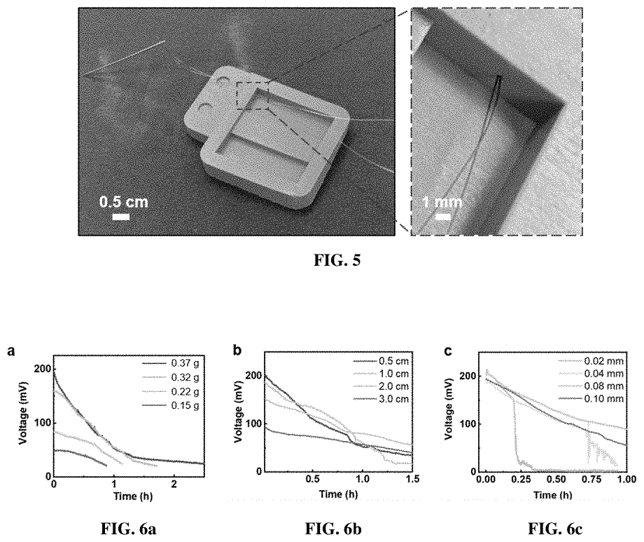

A fabrication process for the battery of FIG. 1(b) is demonstrated in FIG. 4. In this process, a mold is used for forming substrate 10. The impregnated fabric layers are assembled into the substrate 10 along with the cathode and anode, 42, and 44. The absorbent impregnated layer 50 is assembled over the cathode and anode such that the layer 50 will be in contact with the skin of the wearer. The two magnets 20 are embedded in the substrate/carrier 10 in a detachable manner and may be connected to the anode and cathode by conductors. (FIG. 5)

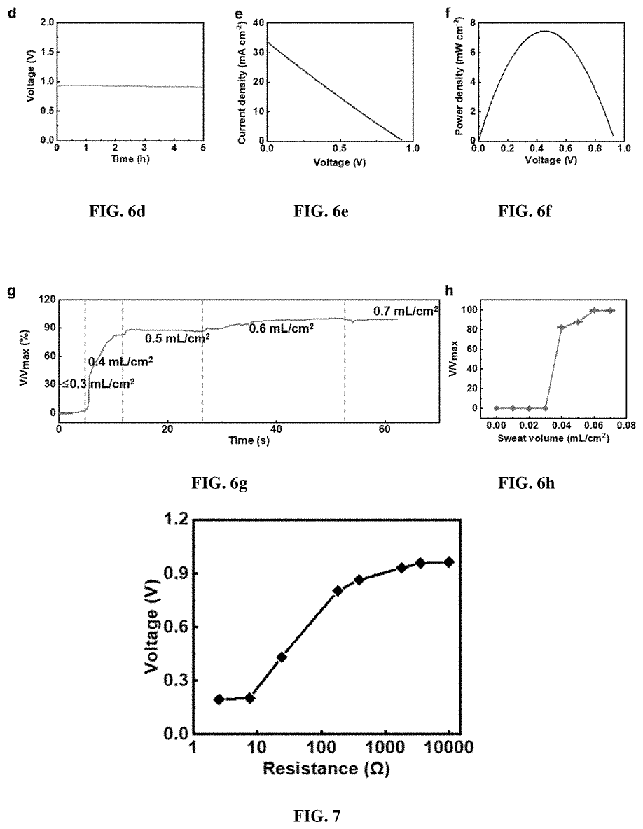

The generated voltage of the battery is measured while it is connected to a 2.5Ω resistor as the load. The generated voltage of the battery on the load depends on the CuSO₄ contents' mass. The higher the CuSO₄ contents are, the higher the voltage is (see, e.g., FIG. 6(a)). The relationship between the output voltage and electrode spacing over a one-and-a-half-hour period is presented in FIG. 6(b). As the distance between two electrodes decreases, the battery voltage increases. However, a larger spacing shows a more stable response over time. The greater the thickness of the Zn foil, the longer the battery life. That is because Zn acts as a reactant in the battery electrolytic reaction and is consumed in the reaction. Using 0.08 mm thick foils, the battery is operable for more than 1 hour. (FIG. 6(c)) As a result, the 0.08 mm thick Zn foil exhibits the best performance and was chosen for the battery. The battery's voltage is stable at 0.97V with a standard deviation of 0.01 in the recorded data for more than 5 hours (FIG. 6(d)). The polarization shows a maximum current density of 33.75 mA.cm⁻². Accordingly, the maximum power density is 7.46 mW.cm⁻², a high output for a sweat-activated battery (FIGS. 6(e, f)).

The output voltage as a proportion of the maximum output voltage, when a controlled volume of artificial sweat is added into the battery, is represented in FIG. 6(g)). The minimum sweat volume (MSV) for activating the SASB was investigated. According to the results, the MSV value is 0.04 mL/cm², yielding ˜82% of the battery's highest open circuit voltage (OCV). 0.05 mL/cm² rate corresponds to the ˜87% OCV, and 0.06 mL/cm² to ˜99% OCV (FIG. 6(h)).

The effect of changing the load resistance is investigated. The output voltage of the battery as a function of the load resistance is presented in FIG. 7. In this FIG., the voltage output of one battery cell is presented as a function of connected resistance from 2.5Ω to 10 kΩ.

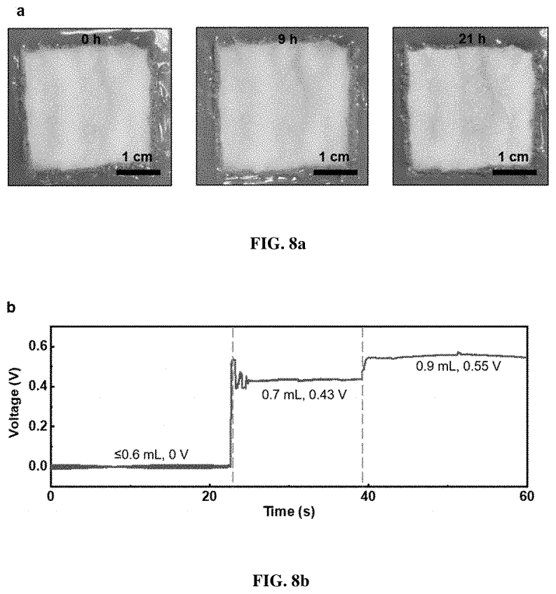

The optical images of the absorbent layer 50 are shown in FIG. 8(a), where 0.6 mL of artificial sweat (CAS, 1336-21-6) is injected. The inventive design successfully prevents the leakage of the inner chemicals during battery usage. The electrical response of the SASB cell without any ionic chemicals in the absorbent layer 50 is plotted as a function of injected artificial sweat (FIG. 8(b)). The highest open-circuit output voltage reaches 0.55 V, far lower than for the KCl impregnated absorbent layer 50. This outcome verifies that a higher concentration of the electrolyte solution (KCl) leads to a higher conductivity within the internal circuit of the battery.

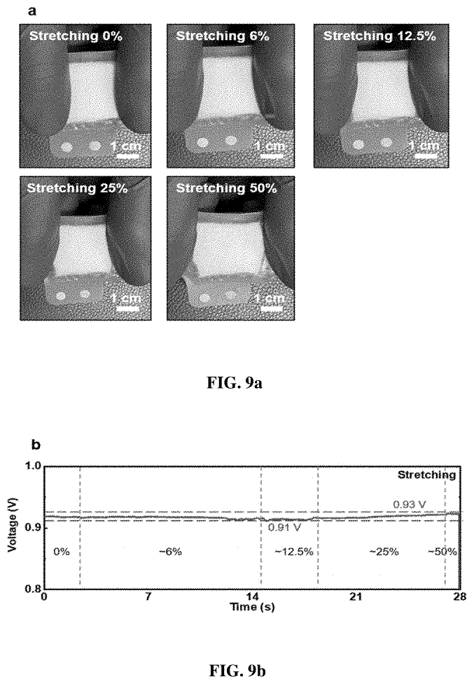

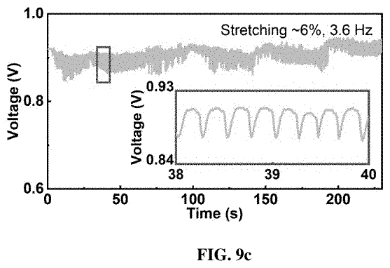

FIG. 9(a) shows the optical images of the SASB under different stretching percentages, including 0, 6%, 12.5%, 25%, and 50%. At the latter stretch rate, the battery starts disassembling. FIG. 9(b) presents the electrical response of the battery under a stretched situation. The OCV slightly fluctuates from 0.91 V to 0.93 V initially, exhibiting a stable performance when it is stretched. The OCV is stable until an approximately 50% elongation, at which point the battery breaks down. In addition, the OCV of the battery stabilizes between 0.85 V and 0.93 V after over 800 cycles at a constant stretching percentage and frequency of ˜6% and 3.6 Hz (FIG. 9(c)).

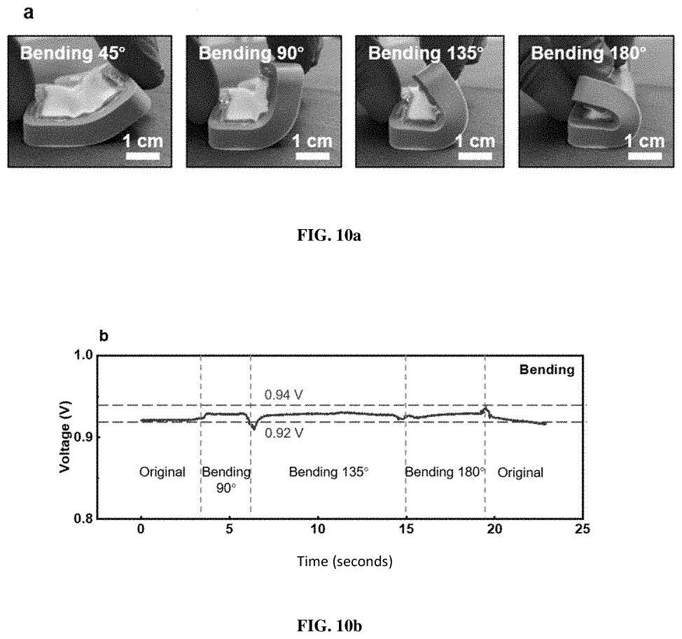

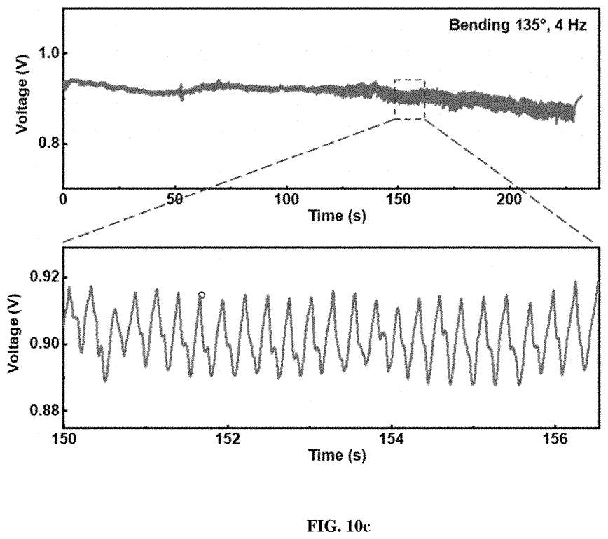

The battery is deformed at different angles, including 45°, 90°, 135°, and 180° (FIG. 10(a)). The open circuit voltage (OCV) is investigated in these conditions, exhibiting a stable output between 0.92 V and 0.94 V (FIG. 10(b)). Furthermore, the battery is deformed at 135° for a longer period of time and shows a stable output, between 0.86 V and 0.93 V at a frequency of 4 (FIG. 10(c)).

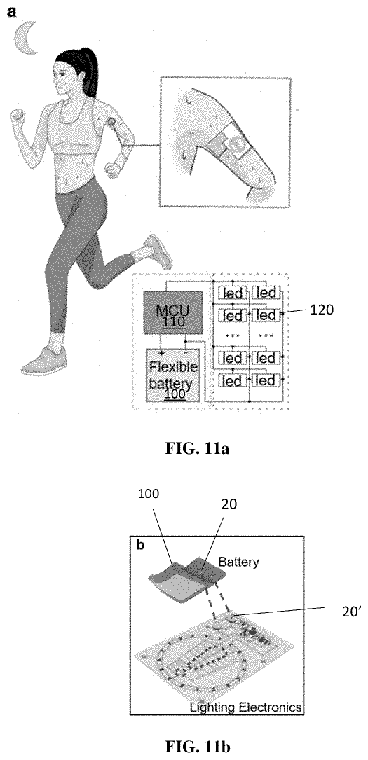

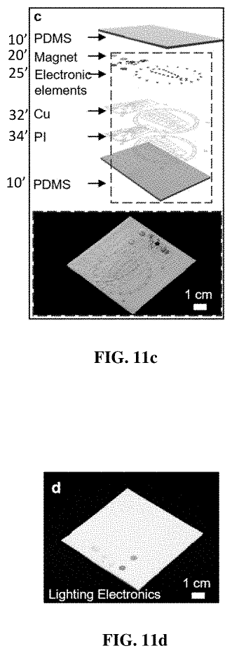

The sweat-activated batteries of the present invention may be used in a variety of flexible and wearable electronic devices. One application of the battery is depicted in FIGS. 11(a)-(d) in which the battery is used to power safety electronics for individuals engaged in outdoor exercise activities. For example, for users exercising at night, the inventive batteries may be used to power flexible lighting electronics attached to the body of a running person. The secreted sweat from the runner activates the battery. As seen in FIG. 11(a), the battery 100 connects to a microcontroller unit 110 using two small magnets 20, which drive current to the lighting LED series 120 on a flexible and stretchable substrate. (FIGS. 11(b-d)) The connection between the battery and flexible lighting electronics is through magnets 20 to 20′ and illustrated in FIG. 11(b). One battery cell provides a high-power density-enough to light 34 green LEDs for more than 8 h (FIGS. 11(f, g)). In addition, the flexibility and portability of the device make it easy to use during running or other physical activities. Due to these characteristics, the device addresses safety concerns for a running individual in the dark by alerting others of his/her presence. In further tests, one battery cell is used for powering 120 green LEDs with a voltage booster on a detachable printed circuit board. In FIG. 11c, a device is shown with PDMS layers 10, cooperating magnets 20′, electronic elements 25 (e.g., lighting LEDs) copper wiring 32′, PI 34′.

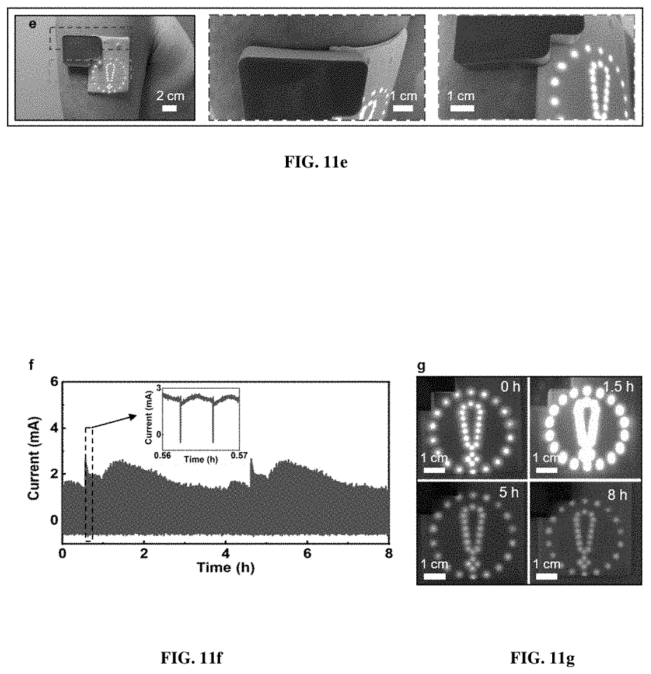

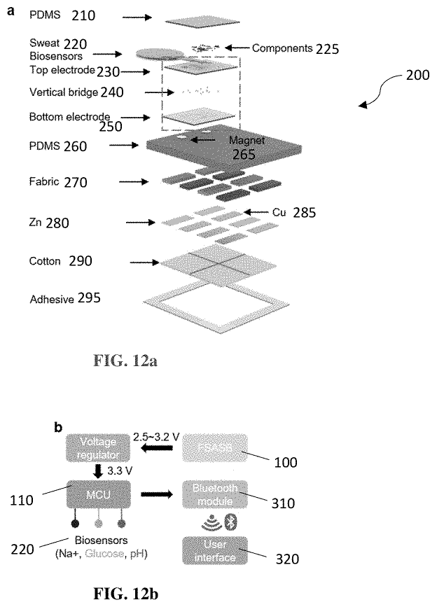

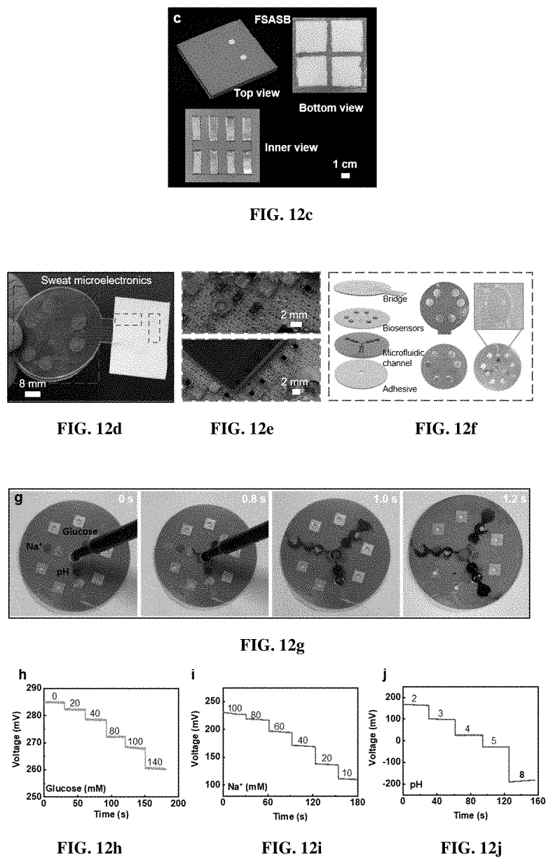

Another application of the SASB is illustrated in FIGS. 12(a)-(j) for monitoring the health status of a user through tracking the concentration of sweat components. There are abundant chemicals in human sweat that reflect the health status. Taking this into consideration, a smart stretchable microelectronic device is developed and powered by four sweat-activated battery cells as described above to wirelessly monitor the concentration of three different sweat biomarkers: pH, Na+, and glucose. The microelectronic device contains a microfluidic system for absorbing human perspiration with a controlled flow rate. The performance of the flexible electronic device containing sensors is examined over different concentrations and over time. Results show stable and accurate measurements. It should be noted that all embedded sensors are designed as potentiometric sensors without a transimpedance amplifier to simplify the circuit design.

FIG. 12(a) shows the schematic diagrams of the four packed sweat-activated stretchable batteries powering the sweat sensor microelectronics (FSASB-SE). As seen in FIG. 12(a), the system 200 includes a PDMS layer 210, sweat biosensors 220, optional electrical components 225, top electrode 230, vertical bridge 240, bottom electrode 250 magnets 265, PDMS layer 260, fabric layer 270, zinc and copper elements 280, 285, absorbent layer (e.g., cotton) 290 and adhesive 295. In another aspect, the system, as shown in FIGS. 12(b, c), includes three parts: 1) a stretchable integrated circuit, incorporating a wireless communication element such as a Bluetooth module 310 (CC2640R2F, Texas Instruments Inc.), for data collection, analysis, and long-range, wireless transmission; 2) Flexible sweat biosensors 220 for pH, Na+, and glucose concentrations measurement and a microfluidic channel for diverting sweat towards the target biosensors with a controlled flow rate and single directional control; 3) plural sweat-activated stretchable battery 100 (FSASB) cells connected in series for providing a high OCV and power density, which is sufficient to power the integrated circuit. Optical images of four battery cells, sweat sensor microelectronic device with detachable microfluidic channels are shown in FIGS. 12(c-f). A process of applying a colored water drop to a microfluidic channel is demonstrated in FIG. 12(g). The output voltage of each sensor for different concentrations of biomarkers shows an accurate and stable response (FIGS. 12(h-j)).

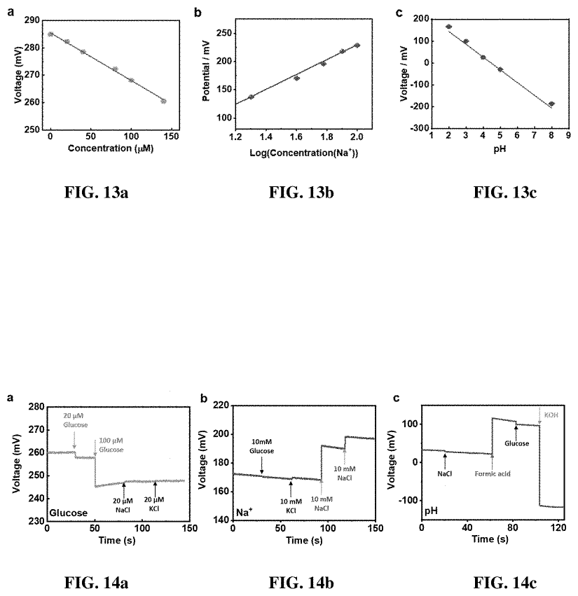

The output voltage of glucose sensors (vs. Ag/AgCl) decreases with the increase of glucose concentration. The voltage (vs. Ag/AgCl) of the glucose sensors is linearly proportional to the glucose concentrations with a determination coefficient (R²) of 0.997. (FIG. 12(h) and FIG. 13(a)) Similarly, the characterization of the Na sensor within physiologically relevant concentrations of 20-100 mM Na is demonstrated, with a good linear relationship between the open-circuit voltage (vs. Ag/AgCl) of the sensor and the value of the logarithm of Na concentration. The determination coefficient (R²) is 0.992, corresponding to a sensitivity of 45.18 mV per decade. (FIG. 12(i) and FIG. 13(b)) Voltage responses and the calibration curve for pH 2 to pH 8 in the electrolyte are presented in FIG. 12(j) and FIG. 13(c). A near-Nernstian sensitivity of 58 mV per pH is observed for the pH sensor based on polyaniline (PANI), with an R² of 0.990. The excellent selectivity and anti-interference characteristics of the three sensors has been tested and verified. (FIG. 14)

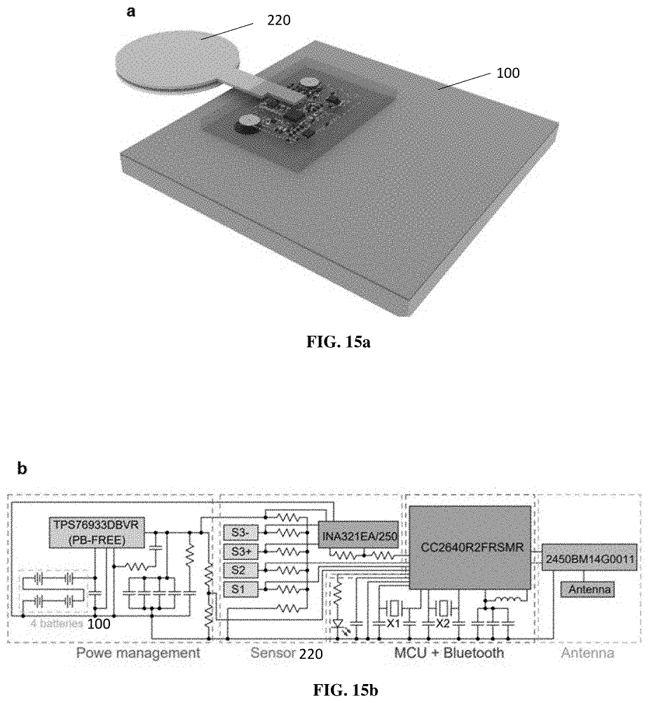

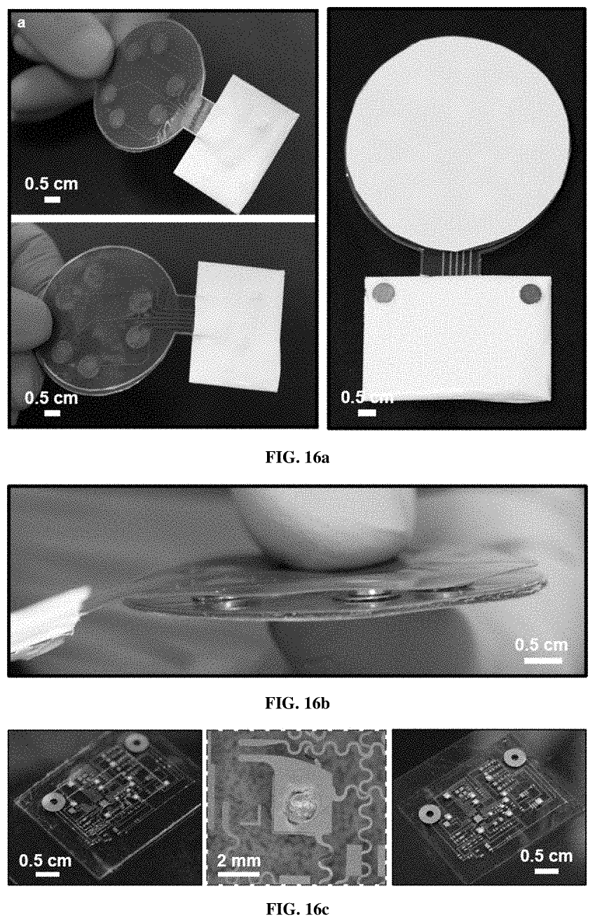

A schematic diagram of the entire sweat microelectronics system and circuit design is shown in FIG. 15(a) and FIG. 15(b), respectively. Additional optical images of the sweat microelectronic device system and the flexible circuit are presented in FIG. 16.

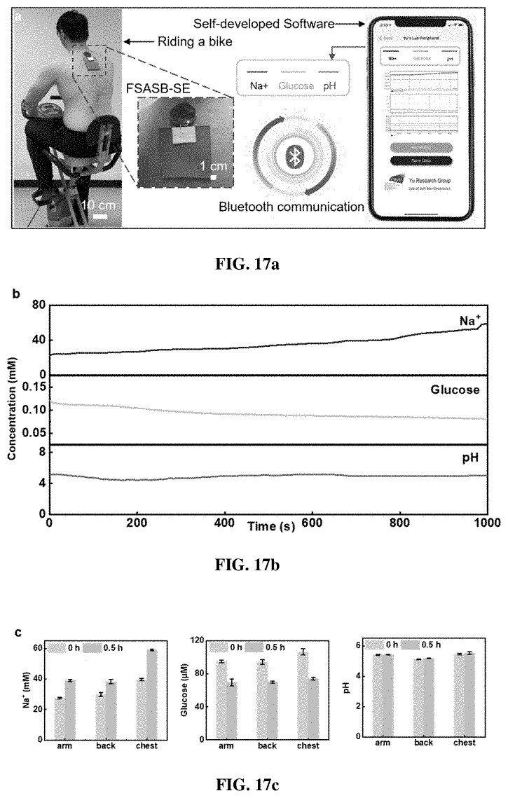

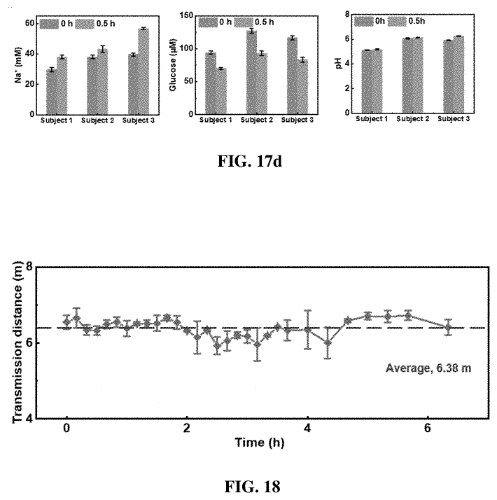

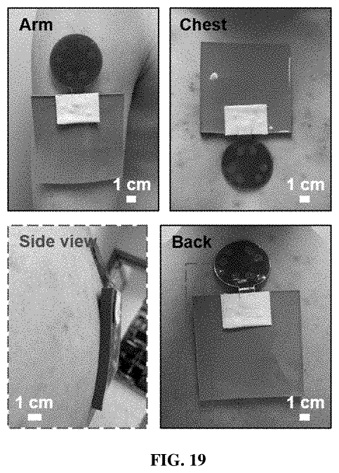

A subject wearing the FSASB-SE system while cycling, and the results displayed on a cell phone is shown in FIG.(a). At the beginning of the test a glucose concentration decreased from 122 mM to 100 mM, then stabilized over time. A stable pH (˜4.9) was obtained throughout the exercise. (FIG. 17(b)) Furthermore, the test has been done by placing the system on different parts of the body, including the arm, back, and chest (FIG. 19). Compared with the initial values, sweat Na⁺ concentration rises, sweat glucose concentration declines, and pH level is stable after 30 minutes of exercising (FIG. 17(c)). Different subjects also have been tested while exercising, and the results of the system are observed. (FIG. 17(d)) Finally, the transmission distance for the wireless monitoring system has been measured for over 6 hours. (FIG. 18) These data indicate that the FSASB-SE may be used in long-term, wireless, personalized physiological signals monitoring.

While the present disclosure has been described and illustrated with reference to specific embodiments thereof, these descriptions and illustrations are not limiting. It should be understood by those skilled in the art that various changes may be made and equivalents may be substituted without departing from the true spirit and scope of the present disclosure as defined by the appended claims. The illustrations may not necessarily be drawn to scale. There may be distinctions between the artistic renditions in the present disclosure and the actual apparatus due to manufacturing processes and tolerances. There may be other embodiments of the present disclosure which are not specifically illustrated. The specification and the drawings are to be regarded as illustrative rather than restrictive. Modifications may be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it will be understood that these operations may be combined, sub-divided, or re-ordered to form an equivalent method without departing from the teachings of the present disclosure. Accordingly, unless specifically indicated herein, the order and grouping of the operations are not limitations.

1. A flexible and stretchable electronic device system comprising;
a sweat-activated battery including:
an anode and a cathode;
a dry electrolyte-impregnated carrier in contact with the anode and the cathode;
a sweat-absorbing layer in contact with the dry electrolyte-impregnated carrier;
an adhesive layer for attaching the sweat-activated battery to the skin of a user;
a flexible electronic device connected to the sweat-activated battery, the flexible electronic device being powered by the sweat-activated battery.

2. The flexible and stretchable electronic device system of claim 1, wherein the dry electrolyte-impregnated carrier includes one or more of CuSO₄ or KCl.

3. The flexible and stretchable electronic device system of claim 1, wherein the sweat-absorbing layer is impregnated with a dry electrolyte.

4. The flexible and stretchable electronic device system of claim 3, wherein the sweat-absorbing layer is impregnated with KCl such that the sweat-absorbing layer acts as a salt bridge in a galvanic cell.

5. The flexible and stretchable electronic device system of claim 1, wherein the flexible electronic device is an LED lighting device.

6. The flexible and stretchable electronic device system of claim 1, wherein the flexible electronic device is a sweat sensor.

7. The flexible and stretchable electronic device system of claim 6, wherein the sweat sensor includes detectors for monitoring pH, sodium levels, and glucose levels.

8. The flexible and stretchable electronic device system of claim 1, further comprising a deformable carrier housing the sweat-activated battery.

9. The flexible and stretchable electronic device system of claim 8, wherein the deformable carrier is polydimethylsiloxane.

10. The flexible and stretchable electronic device system of claim 1 further comprising a wireless transmitter for transmitting signals to a wireless receiver.
